(19) United States
(12) Patent Application Publication
(10) Pub. No.: US 2021/0069001 A1
(43) Pub. Date: Mar. 11, 2021

(54) Zenith Adjustable-Height Cervical Orthotic

(71) Applicant: Diego Caban Delgado, Gurabo, PR (US)

(72) Inventor: Diego Caban Delgado, Gurabo, PR (US)

(21) Appl. No.: 16/562,440

(22) Filed: Sep. 6, 2019

(51) Int. Cl. A61F 5/055

(52) U.S. Cl. CPC A61F 5/055; A47G 2009/1018

(57) ABSTRACT

The present invention discloses an enhanced design of the orthotic device with symmetrical estimations in stature and side width to provide enhanced stability. The assembly has three separate connectable levels which fit as blocks and enable the user to adjust each level as per individual requirements.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or, records, but otherwise reserves all copyright rights whatsoever.

### BACKGROUND

### Field of the Invention

The invention relates to orthotic devices. In a particular form, the present invention relates to attachable levels in curvilinear shape representative of the cervical curve. Directed to a cervical orthotic device for counteracting forward head posture and help the restoration of the proper cervical curve and posture.

### Description of the Related Art

The human neck and back are made up of small bones called vertebrae. These are stacked on top of each other to form the spinal column.

The spinal column supports the head and protects the spinal cord. This is the main structure which links the network of nerves throughout your body. Messages travel along with this network sending sensations, such as pain, to your brain.

The top seven bones in the spinal column form the human neck, and these are called the cervical vertebrae. The bones are linked together by facet joints. These are small joints between your vertebrae that, together with your neck muscles, allow you to move your head in any direction.

Between the vertebrae are discs of cartilage. The discs act as shock absorbers and give the spine its flexibility. A slipped disc occurs when one of these discs slips slightly out of its natural position in the spine.

Neck pain is very common and most of us have it at some point in our lives. Usually, neck pain is the result of holding neck in the same position for too long. However, other things can also cause or contribute to neck pain, such as worry or stress, sleeping awkwardly, an accident, which can cause whiplash, a sprain or a strain, a flare-up of cervical spondylosis; which can happen as the discs and joints in the spine age.

Many people develop a stiff and painful neck for no obvious reason. It may happen after sitting in a draught or after a minor twisting injury, for example, while gardening. This is called non-specific neck pain. This is the most common type of neck pain and usually disappears after a few days, providing you keep gently moving your neck and rest when you need to.

Multiple solutions that have been presented toward managing this situation. For instance, Neck Orthotic, Orthotic pillows, massage tools and other medical assemblies. Similarly, in prior art, we can also see multiple inventions with their own set of advancement.

A Backbone correction exercise apparatus bearing U.S. Pat. No. 7,637,854B2 is issued to Suk Hwan Ring. The patent discloses a backbone correction exercise apparatus is provided, in which a user lies on a bed and wears a pelvic belt, adjusts distance from an exercise unit depending on a user's physical condition, and then perform recursive exercises, to thereby slack and restore the cervical vertebra portion and the lumbar vertebra portion of the human body repeatedly and to thus strengthen spinal peripheral support muscles in order to provide a spinal curative effect as well as a spinal exercise effect, and which includes and exercise unit which enables a user to lie on a bed and take an exercise using the user's feet, and a pelvis belt whose intermediate portion is fixed to the bed on which the user lies and whose side extensions extended from the intermediate portion rises from the bed and wrap and hold a portion corresponding to the waist of the user with a binding unit which is provided in the ends of the extensions.

Another patent bearing U.S. Pat. No. 7,086,992B2 is issued to Jason Bowman Robert Gearhart Robert L. Richardson. The patent discusses a posture correction exercise device is disclosed to aid in correcting the common postural condition of kyphosis-lordosis by aiding in the exercise of the spinal erectors to strengthen the erectors to pull the user's spine and torso backward into normal alignment and by exercise of the mid-trapezius, rhomboid and posterior deltoid muscles to strengthen these muscles to pull the user's shoulder blades together and force the shoulders into normal alignment. The device operates by seating the user upon a declined seat to provide increased resistance by gravity. The device provides resistance to backward movement of user's body and resistance to backward rotation of user's arms. The hands of the user are positioned in supinated palms-up hand positions to increase the training effect of the backward rotation of the user's arms.

Another Head, neck and shoulder therapeutic exercise device bearing U.S. Pat. No. 5,116,359A is issued to MOORE JOSEPH L. The patent is about a head, neck and shoulder therapeutic exercise device which mechanically assists the human head and neck with intermittent rearward/forward movement in order to help correct or attenuate by means of muscular conditioning the cervical misalignment associated with forward head and flexible cervical lordosis. Mounted on a chair, an adjustable frame supports above the patient's head a motor assembly in fixed position and a motion-assist assembly which is horizontally moveable. The latter comprises in part a roller block with affixed roller bearing plate extending down behind the patient's head. The motor shaft, also behind the patient's head, has a cam which rides against a roller bearing in the roller bearing plate. As the cam's high side rotates against the bearing, the bearing plate moves away from the patient, thereby transferring rearward tension to the head using stretchable straps attached to said bearing plate and placed around the patient's forehead and chin. Each rotation of the cam is briefly halted by a timer when the head is in retracted position. After a holding phase, the rotation resumes, returning everything to original position. The head displacement distance is controlled by variously sized interchangeable cams. During therapy, the patient tries to do chin tuck exercises to accompany the cyclic mechanical movement.

Another Head and neck cushion bearing U.S. Pat. No. 4,494,261A is issued to WINSTON MORROW. The patent is about a composite head and neck cushion is provided for use by a person in a supine position. The cushion includes a first, resilient member having an upper surface which conforms to and supports the physiologic curvature of the cervical vertebrae. A second member supports the head in a raised, but unflexed position. The invention may alternatively be described as a method for cushioning the head and neck. The method comprises (l) resiliently supporting the back of the neck of the person in an elevated position while permitting the cervical vertebrae to maintain their normal, physiologic curvature, and (2) supporting the head of the person in an elevated position while maintaining the cervical vertebrae and the occiput in an unflexed, physiologic position.

A Cervical/occipital support bearing U.S. Pat. No. 4,708,129A is issued to Charles Pujals, Jr. The patent is about a one-piece contoured neck brace or support is disclosed which provides semi-rigid support for a user's head and neck by conforming to and contacting the occiput between the ears, the posterior and lateral neck area and the suprascapular region. The support may be retained on the user's neck by an adjustable strap or collar, and has forwardly extending portions to reduce any uncomfortable pressure on the user's throat. Ear protectors may be selectively attached to the support as needed, as may a headband. The headband may be used to further secure the support, the ear protectors, or both.

A Cervical pillow bearing U.S. Pat. No. 4,424,599A is issued to Nabil Hannouche. The patent is about a cervical pillow having a shoulder engaging portion, an occiput engaging portion, and a neck engaging portion therebetween. The cervical pillow provides continuous support from the shoulder portion to the occipital portion of the user.

A Cervical/occipital support bearing U.S. Pat. No. 4,854,306A is issued to Charles Pujals, Jr. The patent is about support is provided for a user's head and neck which provides varying degrees of support depending on the combination of support members utilized. A posterior shell which supports the occiput can be combined with either ear protectors or an anterior shell, or both and an overlying posterior shell can be utilized to provide increased support. A single posterior shell could include all support features such as the ear protectors formed as an integral part thereof. The anterior shell can have molded contours to increase its rigidity and jaw engaging sidewalls to reduce lateral movement.

A U.S. Pat. No. 4,793,334 also discusses neck support which incorporates a system and ties for tying down the structure to the body. A jawline bolster part is situated with respect to the structure to display development of the neck. The jawline bolster part is upheld on a swagger. The swagger is releasably and critically mounted to the system. A prop part stretching out from the swagger gives pivotable development of the swagger.

Multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them not suitable for every type of user. Moreover, the ease of use ability of these assemblies is also questionable.

It has to be noted that the current invention proposes an assembly with multiple advancements including a structure with symmetrical measurements in height and side width that make it more stable and easier to wear for the user. The assembly has three completely separate but attachable levels in curvilinear shape that allow the user to adapt each level to their specific need without having to but different sized blocks or cut through these. The assembly further has a dent/cut in the front and back to place the shoulders and base of the head. The assembly is made of high-density EVA foam to allow for changes to occur over time.

None of the previous inventions and patents, taken either singly or in combination, is seen to describe the instant invention as claimed. Hence, the inventor of the present invention proposes to resolve and surmount existent technical difficulties to eliminate the aforementioned shortcomings of the prior art.

### SUMMARY

In light of the disadvantages of the prior art, the following summary s provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a cervical orthotic used to elicit changes in soft and hard tissues over time.

It is another objective of the invention to provide an orthotic device which being used is increasingly agreeable for the patient.

It is also the objective of the invention to provide a device of the type described which can be easily be adjusted to the fit accomplish the need.

It is another objective of the invention to provide a device that can be used for a variety of factors but not limited to those factors resulting from looking down at hand-held devices, sleeping with the head elevated excessively, or an acute injury such as whiplash.

It is further the objective of the invention that the block has three completely, separate levels of care that do not require cutting through each level to use it. The user just removes the upper levels as needed to use the levels underneath.

A further object is to provide an assembly that is simple, rapidly adjusted and easy to use.

A still further object is to use design and functional materials that allow for the decrease in production cost and price to the patients.

Other aspects, advantages and novel features of the present invention will become apparent from the detailed description of the invention when considered in conjunction with the accompanying drawings.

This Summary is provided merely for purposes of summarizing some example embodiments, to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 100 is a top view thereof of the Cervical Orthotic Device;

FIG. 200 shows three completely separate but attachable levels in curvilinear shape.

FIG. 300 is a close side view of the Cervical Orthotic Device.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is to describe particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to a cervical orthotic used to elicit changes in soft and hard tissues over time.

Whether the pain is caused by a car accident, a fall, or simply poor posture, abnormal cervical lordosis can adversely affect the nerve, ligament, and muscle function. As shown in numerous research studies, those adverse effects can be reversed by correcting cervical lordosis, relieving pain and improving overall health.

A simple orthotic device is engineered with curves, angles and ridges which is symmetrical in measurement. The symmetrical sides both horizontally and vertically allow a stable assembly.

The device, as per its further embodiments, allows three completely separate but attachable levels in curvilinear shape. These levels allow the user to adapt the block to their specific need without having to use different sized blocks or cut through the levels.

The assembly, as per its further embodiments, has an identical dent/cut in the front and back to place the shoulders and base of the head on each of these. This is done to increase the ergonomics of use and to be able to rest the base of the head further down and behind on the block, placing more stretch on the cervical anatomical elements.

After receiving simple training and education, the patient will be able to properly position the device for appropriate use, and consistent use at home.

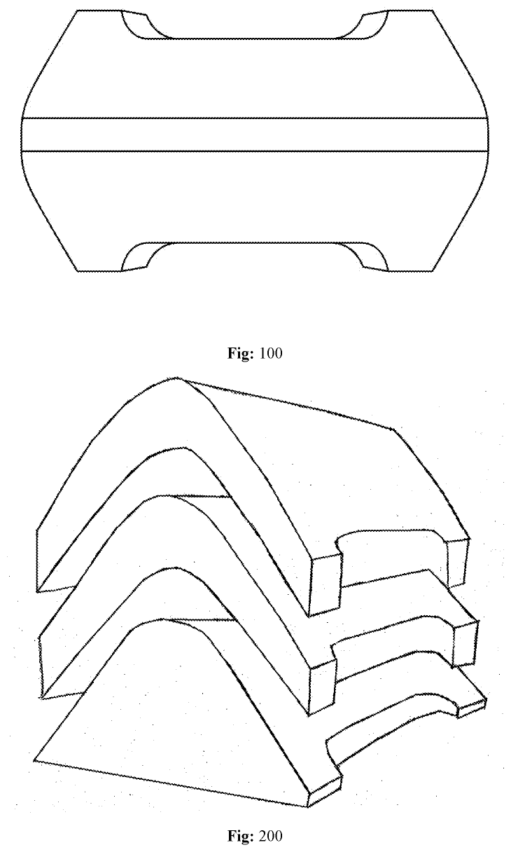

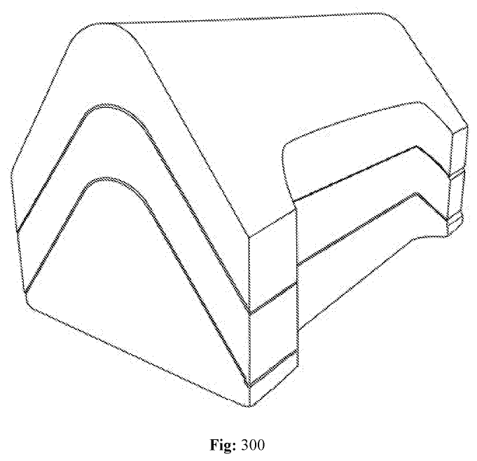

To explain this further a reference with drawings is attached. FIGS. 100 to 300 show an example of an orthotic device according to a first preferred embodiment of the present invention. The orthotic device has three completely independent levels of care that do not require cutting through each level to use it. The user just removes the upper levels as needed to use the levels underneath. It is possible to move up and down from or to each level at any time.

The device is designed symmetrically to be used reversibly at the from or back end of the longitudinal side. For smaller patients or less aggressive treatment, the user carefully removes sections by simply lifting the upper level of high-density EVA foam.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

I: An adjustable cervical orthotic apparatus comprising:
a) a body having symmetrical design from width and length, inferior and superior view;
b) planar surface measuring same from midline;
c) two superior planar surfaces stemming downwardly from the arcuate midline portion to both, longitudinally located, opposing sides;
d) three completely detachable and attachable levels of care that do not require cutting through these. Each following the same curvilinear surface shape;
e) equal side width (depth) and height for enhanced stability;
f) base with a planar surface;
g) all three levels of support made of high-density EVA foam;
h) symmetrical curved dent in the front and back of the block to comfortably place the shoulders and rest the base of the head.

II: An orthotic device having three completely separate levels of care allowing the user to just remove the upper levels as needed to use the levels underneath, Each level follows the same curvilinear shape superiorly.

III: An orthotic block of claim 1, wherein the surface area of the first support surface comes in contact with the patient's neck and the base area is in contact with the substrate surface, the block has three completely separate levels of care that do not require cutting through each level to use it the user just removes the upper levels as needed to use the levels underneath.
